(19) United States
(12) Patent Application Publication
(10) Pub. No.: US 2025/0108203 A1
(43) Pub. Date: Apr. 3, 2025

(54) TRANSDUCER ARRAY HAVING SUBARRAYS AND METHODS OF PRODUCTION AND USE THEREOF

(71) Applicant: Novocure GmbH, Root D4 (CH)

(72) Inventors: Christopher H. Scholl, West Chester, PA (US); Eric C. Sugalski, Berwyn, PA (US)

(21) Appl. No.: 18/899,709

(22) Filed: Sep. 27, 2024

Related U.S. Application Data
(60) Provisional application No. 63/586,260, filed on Sep. 28, 2023.

(51) Int. Cl. A61N 1/04; A61N 1/36

(52) U.S. Cl. CPC A61N 1/0476; A61N 1/36002; A61N 1/36034

(57) ABSTRACT

A transducer array is herein disclosed. The transducer array comprises a hub and a transducer subarray. The hub comprises a housing having a housing peripheral edge having a boundary, and a hub port supported by the housing, wherein the hub port is positioned within the boundary of the housing peripheral edge. The transducer subarray comprises an electrode, a subarray port, and a support layer having a support layer peripheral edge and supporting the electrode and the subarray port. The subarray port couples the hub port and the electrode, and the electrode receives an alternating current waveform having a frequency in a range between 50 kHz-1 MHz from the hub via the subarray port.

### REFERENCE TO RELATED APPLICATIONS

The present patent application claims priority to the United States provisional patent application identified by U.S. Ser. No. 63/586,260, filed on Sep. 28, 2023, the entire content of which is hereby incorporated herein by reference.

### BACKGROUND

Tumor Treating Fields (TTFields) are low intensity (e.g., 1-3 V/cm) alternating electric fields within the intermediate frequency range (e.g., 50 kHz to 1 MHZ, such as 50-500 kHz) that target solid tumors by disrupting mitosis. This non-invasive treatment targets solid tumors and is described, for example, in U.S. Pat. Nos. 7,016,725; 7,089,054; 7,333,852; 7,565,205; 8,244,345; 8,715,203; 8,764,675; 10,188,851; and 10,441,776. TTFields are typically delivered through two pairs of transducer arrays that generate perpendicular fields within the treated tumor; the transducer arrays that make up each of these pairs are positioned on opposite sides of the body part that is being treated. More specifically, for the OPTUNE® system, one pair of electrodes of the transducer array is located to the left and right (LR) of the tumor, and the other pair of electrodes of the transducer array is located anterior and posterior (AP) to the tumor. TTFields are approved for the treatment of glioblastoma multiforme (GBM), and may be delivered, for example, via the OPTUNE® system (Novocure Limited, St. Helier, Jersey), which includes transducer arrays placed on the patient's shaved head. More recently, TTFields therapy has been approved as a concomitant therapy with chemotherapy for malignant pleural mesothelioma (MPM), and may find use in treating tumors in other parts of the body.

The transducer arrays having a hydrogel layer are placed on the patient's skin on opposite sides of a target location (or locations) determined to have a high therapeutic value to treat the patient. The device is intended to be continuously worn by the patient for a wear period of 2-4 days before removal for hygienic care and re-shaving (if necessary), followed by reapplication with a new set of arrays.

As people move, the connecting cables can shift the array away from the target location, thereby decreasing the therapeutic value. Additionally, because the arrays have to be worn for an extended period of time, they can become uncomfortable due to the rigidity of the current arrays. When a target area is near an avoidance area (such as, for example, a chemotherapy port, a nipple, etc.), traditional arrays may not be configured for comfortable placement so as to not interfere and/or overlap with the avoidance area.

### SUMMARY OF THE DISCLOSURE

Thus, a need exists for a new and improved transducer array that is configured to maintain position while improving comfortability for the patient. It is to such systems and methods of producing and using the same, that the present disclosure is directed.

The problem of maintaining position while improving comfortability for the patient is solved by a transducer array, a tumor treating field system, and method of production and use thereof as described herein. In one embodiment, the transducer array comprises a hub and a transducer subarray. The hub comprises a housing having a housing peripheral edge having a boundary, and a hub port supported by the housing, wherein the hub port is positioned within the boundary of the housing peripheral edge. The transducer subarray comprises an electrode, a subarray port, and a support layer having a support layer peripheral edge and supporting the electrode and the subarray port. The subarray port couples the hub port and the electrode, and the electrode receives an alternating current waveform having a frequency in a range between 50 kHz-1 MHz from the hub via the subarray port.

In another embodiment, the transducer array comprises a plurality of electrode elements, a first transducer subarray, a second transducer subarray and a flexible cable. The plurality of electrode elements comprises an electrode configured for placement on a body of a patient and to receive an alternating current waveform. The first transducer subarray comprises a first support layer having a first support layer peripheral edge and supporting a first subset of the plurality of electrode elements operable to receive the alternating current waveform. The second transducer subarray comprises a second support layer having a second support layer peripheral edge and supporting a second subset of the plurality of electrode elements operable to receive the alternating current waveform, the second support layer peripheral edge being disposed apart from the first support layer peripheral edge. The flexible cable is electrically coupled to the first subset of the plurality of electrode elements and operable to transmit the alternating current waveform to the second subset of the plurality of electrode elements.

### BRIEF DESCRIPTION OF THE DRAWINGS

The accompanying drawings, which are incorporated in and constitute a part of this specification, illustrate one or more implementation described herein. The drawings are not intended to be drawn to scale, and certain features and certain views of the figures may be shown exaggerated, to scale or in schematic in the interest of clarity and conciseness. Not every component may be labeled in every drawing. Like reference numerals in the figures may represent and refer to the same or similar element or function and a detailed description of like reference numerals varying only in alphabetical suffix may be omitted for conciseness unless otherwise specified. In the drawings:

FIG. 1 is an exemplary embodiment of a schematic diagram of electrodes as applied to living tissue;

FIG. 2 is an exemplary embodiment of an electronic device configured to generate an alternating electric field constructed in accordance with the present disclosure;

FIG. 3A is a diagram of an exemplary embodiment of a transducer array constructed in accordance with the present disclosure;

FIG. 3B is a cross-sectional diagram of the exemplary embodiment of the hub of FIG. 3A along the line 3B-3B′ constructed in accordance with the present disclosure;

FIG. 4 is a cross-sectional view of an exemplary embodiment of the electrode element of the first transducer subarray of FIG. 3A along line 4-4′;

FIG. 5 is a front view of an exemplary embodiment of the electronic apparatus of FIG. 2 constructed and used in accordance with the present disclosure;

FIG. 6A is a perspective view of another embodiment of a transducer array constructed in accordance with the present disclosure;

FIG. 6B is a diagram of the transducer array of FIG. 6A, constructed in accordance with the present disclosure; and

FIG. 7 is a process flow diagram of an exemplary embodiment of a process of using the electronic apparatus and the transducer array(s) of FIG. 2 to apply an alternating electric field to a patient in accordance with the present disclosure.

### DETAILED DESCRIPTION

Before explaining at least one embodiment of the inventive concept(s) in detail by way of exemplary language and results, it is to be understood that the inventive concept(s) is not limited in its application to the details of construction and the arrangement of the components set forth in the following description. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Headings are provided for convenience only and are not to be construed to limit the disclosure in any manner. Embodiments illustrated under any heading or in any portion of the disclosure may be combined with embodiments illustrated under the same or any other heading or other portion of the disclosure. Any combination of the elements described herein in all possible variations thereof is encompassed by the disclosure unless otherwise indicated herein or otherwise clearly contradicted by context. Unless otherwise required by context, singular terms shall include pluralities and plural terms shall include the singular.

All of the compositions, assemblies, systems, kits, and/or methods disclosed herein can be made and executed without undue experimentation in light of the present disclosure. Where a method claim does not specifically state in the claims or description that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, or the number or type of embodiments described in the specification.

The use of the term “a” or “an” when used in conjunction with the term “comprising” in the claims and/or the specification may mean “one,” but is also consistent with “one or more,” “at least one,” and “one or more than one.” The term “plurality” refers to “two or more.” The use of the term “or” in the claims is used to mean an inclusive “and/or” unless explicitly indicated to refer to alternatives only or unless the alternatives are mutually exclusive.

In addition, the use of the term “at least one of X, Y, and Z” will be understood to include X alone, Y alone, and Z alone, as well as any combination of X, Y, and Z. The use of ordinal number terminology (e.g., “first,” “second,” “third,” “fourth,” etc.) is solely for the purpose of differentiating between two or more items and is not meant to imply any sequence or order or importance.

Circuitry, as used herein, may be analog and/or digital components, or one or more suitably programmed processors (e.g., microprocessors) and associated hardware and software, or hardwired logic. Also, “components” may perform one or more functions. The term “component,” may include hardware, such as a processor (e.g., microprocessor), an application specific integrated circuit (ASIC), a field programmable gate array (FPGA), a combination of hardware and software, and/or the like. The term “processor” as used herein means a single processor or multiple processors working independently or together to perform a task.

As used herein, the term TTField (or TTFields) refers to low intensity (e.g., 1-4 V/cm) alternating electric fields of medium frequencies (about 50 kHz-1 MHZ, and more preferably from about 50 kHz-500 kHz) that when applied to a conductive medium, such as a human body, via electrodes may be used, for example, to treat tumors as described in U.S. Pat. Nos. 7,016,725; 7,089,054; 7,333,852; 7,565,205; 8,244,345; 8,715,203; 8,764,675; 10,188,851; and 10,441,776 by Palti, the entire contents of which are hereby incorporated herein in their entirety, and in a publication by Kirson (see Eilon D. Kirson, et al., Disruption of Cancer Cell Replication by Alternating Electric Fields, Cancer Res. 2004 64:3288-3295). TTFields have been shown to have the capability to specifically affect cancer cells and serve, among other uses, for treating cancer. TTFields therapy is an approved mono-treatment for recurrent glioblastoma (GBM), and an approved combination therapy with chemotherapy for newly diagnosed GBM patients. Alternating electric fields can also be used to treat medical conditions other than tumors.

As used herein, the term TTSignal is an electrical signal that, when received by electrodes applied to a conductive medium, such as a human body, causes the electrodes to generate the TTField described above. The TTSignal is often an AC, or alternating current, electrical signal.

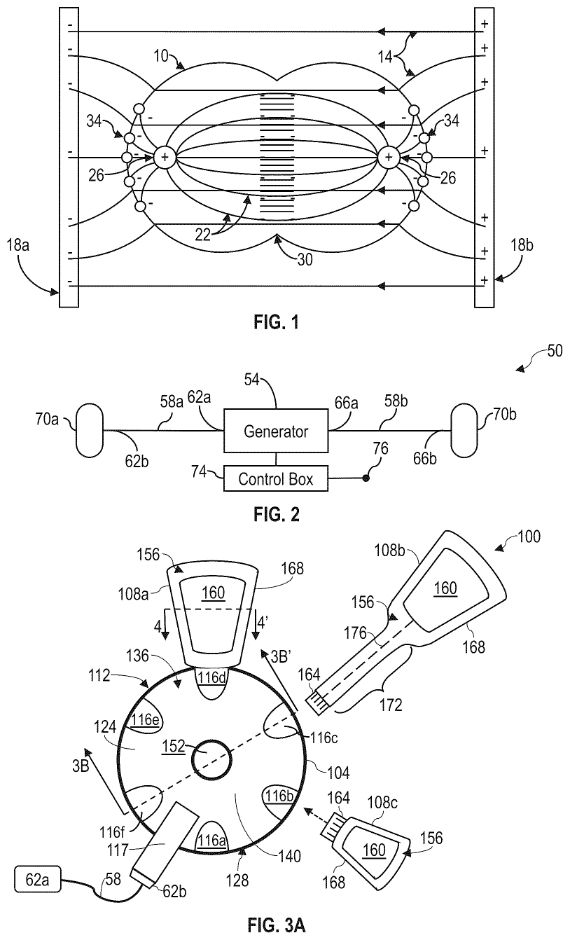

Referring now to the drawings and in particular to FIG. 1, shown therein is an exemplary embodiment of a dividing cell 10, under the influence of external TTFields, generally indicated as lines 14, generated by a first electrode 18a having a negative charge and a second electrode 18b having a positive charge. Further shown are microtubules 22 that are known to have a very strong dipole moment. This strong polarization makes the microtubules 22, as well as other polar macromolecules and especially those that have a specific orientation within the cell 10 or its surroundings, susceptible to electric fields. The positive charges of the microtubules 22 are located at two centrioles 26 while two sets of negative poles are at a center 30 of the dividing cell 10 and point of attachment 34 of the microtubules 22 to the cell membrane. The locations of the charges form sets of double dipoles and therefore are susceptible to electric fields of differing directions.

Turning now to FIG. 2, the TTFields described above that have been found to advantageously destroy tumor cells may be generated by an electronic apparatus 50. FIG. 2 is a simple schematic diagram of the electronic apparatus 50 illustrating major components thereof. The electronic apparatus 50 includes an electric field generator 54 and a pair of conductive leads 58, including first conductive lead 58a and second conductive lead 58b. The first conductive lead 58a includes a first end 62a and a second end 62b. The second conductive lead 58b includes a first end 66a and a second end 66b. The first end 62a of the first conductive lead 58a is conductively attached to the electric field generator 54 and the first end 66a of the second conductive lead 58b is conductively attached to the electric field generator 54. The conductive leads 58 are isolated conductors with a flexible metal shield, preferably grounded thereby preventing spread of any electric field generated by the conductive leads 58.

The electric field generator 54 is configured to supply power and generate desirable electric signals (TTSignals) in the shape of waveforms or trains of pulses as an output. The second end 62b of the first conductive lead 58a is connected to a first transducer array 70a and the second end 66b of the second conductive lead 58b is connected to a second transducer array 70b. Both of the first transducer array 70a and the second transducer array 70b are supplied with the electric signals (e.g., TTSignals, wave forms). The first transducer array 70a and the second transducer array 70b, being supplied with the electric signals, causes an electrical current to flow between the first transducer array 70a and the second transducer array 70b. The electrical current generates an electric field (i.e., TTField), having a frequency and an amplitude, to be generated between the first transducer array 70a and the second transducer array 70b.

While the electronic apparatus 50 shown in FIG. 2 comprises only two transducer arrays 70 (i.e., the first transducer array 70a and the second transducer array 70b), in some embodiments, the electronic apparatus 50 may comprise more than two transducer arrays 70.

The electric field generator 54 generates an alternating voltage wave form (i.e., TTSignal) at frequencies in the range from about 50 kHz to about 1 MHz (preferably from about 100 kHz to about 500 kHz). The required voltages are such that an electric field intensity in tissue within the treatment area is in the range of about 0.1 V/cm to about 10 V/cm.

In certain particular (but non-limiting) embodiments, the first transducer array 70a and the second transducer array 70b generate an alternating electric current and field within a target region of a patient. The target region may comprise at least one tumor (or region including a resection cavity after removal of the tumor), in which case the generation of the alternating electric current and field selectively destroys and/or inhibits growth of the tumor (or cancer cells). The alternating electric current and field may be generated at any frequency suitable for treating a patient, for example that selectively destroys or inhibits growth of the tumor (or cancer cells), such as at any frequency of a TTField.

In order to optimize the electric field (e.g., TTField) distribution, the first transducer array 70a and the second transducer array 70b (pair of transducer arrays 70) may be configured differently depending upon the application in which the pair of transducer arrays 70 are to be used. The pair of transducer arrays 70, as described herein, are externally applied to a patient in order to apply the electric current, and electric field (e.g., TTField) thereby generating current within the patient's tissue. Generally, the pair of transducer arrays 70 are placed on the patient's skin by a user such that the electric field is generated across patient tissue within a treatment area. Alternating electric fields that are applied externally can be of a local type or widely distributed type, for example, the treatment of skin tumors and treatment of lesions close to the skin surface.

In one embodiment, the user may be a medical professional, such as a doctor, nurse, therapist, or other person acting under the instruction of a doctor, nurse, or therapist. In another embodiment, the user may be the patient, that is, the patient (and/or a helper) may place the first transducer array 70a and the second transducer array 70b on the patient's treatment area.

According to another exemplary embodiment, the electronic apparatus 50 includes a controller 74. In one embodiment, the controller 74 comprises circuitry configured to control the output of the electric field generator 54, for example, to set the output at the maximal value that does not cause excessive heating of the treatment area. The controller 74 may issue a warning, or the like, when a temperature of the treatment area exceeds a preset limit. A temperature sensor 76 may be mechanically connected to and/or otherwise associated with the first transducer array 70a and/or the second transducer array 70b so as to sense the temperature of the treatment area at either one or both of the first transducer array 70a or the second transducer array 70b.

In some embodiments, the electric field generator 54 may be configured to transmit the TTSignal and/or TTField wirelessly thereby removing the need for the conductive leads 58 to provide a direct electrical connection between the electric field generator 54 and the transducer array 70. Exemplary embodiment of a wireless connection may be constructed in accordance any of the embodiments disclosed in U.S. Provisional Application No. 63/387,113, filed on Dec. 13, 2022, and entitled “WIRELESS TRANSDUCER ARRAYS APPLYING TUMOR TREATING FIELDS AND SYSTEMS AND METHODS OF USE THEREOF,” the entire contents of which are incorporated by reference herein, in its entirety.

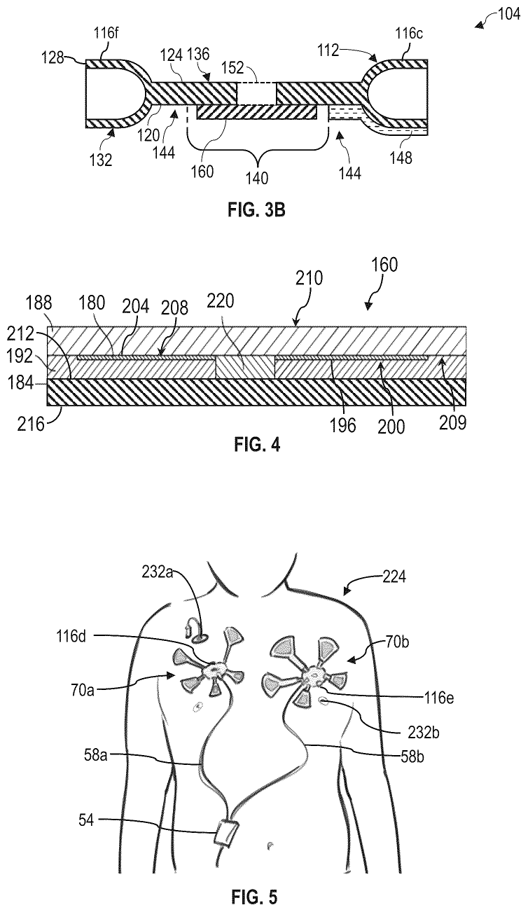

Referring now to FIGS. 3A-3B, in combination, shown in FIG. 3A is a diagram of an exemplary embodiment of a transducer array 100 constructed in accordance with the present disclosure, and FIG. 3B is a cross-sectional diagram of the hub of the exemplary embodiment of FIG. 3A along the line 3B-3B′. The transducer array 100 may be a particular embodiment of the transducer array 70 (FIG. 2). The transducer array 100 generally comprises a hub 104 and one or more transducer subarray 108, shown as a first transducer subarray 108a, a second transducer subarray 108b, and a third transducer subarray 108c. While the transducer array 100 is shown having three transducer subarrays 108a-c, it will be understood that the transducer array 100 may include as few as one transducer subarray 108 or as many transducer subarrays 108 as are able to provide a therapeutic benefit or as may be coupled to the hub 104.

In one embodiment, the hub 104 includes a housing 112 supporting a plurality of hub ports 116 (hereinafter hub ports 116, shown in FIG. 3A as hub ports 116a-f) as well as a hub lead connector port 117 which may be configured to receive at least one end of the conductive lead 58. The housing 112 comprises a first surface 120 on a skin-facing side 132 (shown in FIG. 3B), a second surface 124 on an opposing outwardly-facing side 136, and a housing peripheral edge 128 thereby forming a boundary. In one embodiment, the housing 112 (optionally) includes a passage 152 extending from the first surface 120 to the second surface 124, thereby providing a pathway for air to flow to the patient's skin when the hub 104 is applied to the patient, thus allowing the air to cool the patient's skin and/or provide a pathway for sweat.

In one embodiment, a first number, n, of the hub ports 116 supported by the housing 112 may be positioned within the boundary of the housing peripheral edge 128. Generally, the hub ports 116 may be disposed between the first surface 120 and the second surface 124; however, it will be understood that in some embodiments, at least a portion of the hub ports 116 may extend into the first surface 120 or the second surface 124. The hub ports 116 may be equally disposed about the boundary of the housing peripheral edge 128 (e.g., equidistant from one another, or approximately equidistant), as shown in the exemplary embodiment of FIG. 3A, or the hub ports 116 may have a non-uniform distribution about the housing peripheral edge 128. In one embodiment, n transducer subarrays are electrically coupled to all of the respective n hub ports 116 via a respective subarray port 164 (described below). In one embodiment, n-1 or n-2 transducer subarrays may be coupled to a respective one of the first number, n, hub ports 116 via a respective subarray port 164.

In one embodiment, the hub lead connector port 117, may be configured to receive at least one end of the conductive lead 58. For example, the hub lead connector port 117 may be configured to receive the second end 62b of the conductive lead 58 (which in turn is connected to the electric field generator 54 via the first end 62a of the conductive lead 58), thereby supplying the TTSignal to all of the hub ports 116. In one embodiment, the second end 62b may be fixedly attached to the hub lead connector port 117; alternatively, the second end 62b may be selectively attached to the hub lead connector port 117. In one embodiment, the second end 62b of the conductive lead 58 may comprise a second port operable to be connected and/or coupled to the hub lead connector port 117.

In one embodiment, the hub ports 116 may have a port type, such as a standardized port, a non-standardized port, or a combination of non-standardized and standardized ports. For example, the hub ports 116 (and, optionally, the hub lead connector port 117) may include, but are not limited to: a USB type-C type, serial port type, parallel port type, USB type, and combinations thereof, or the like. Exemplary embodiments of hub ports 116 may be constructed in accordance with any of the connectors disclosed in U.S. application Ser. No. 17/490,120 filed on Sep. 30, 2021, and entitled “CONNECTOR FOR DETACHABLE ARRAY,” the entire contents of which is incorporated by reference.

In one embodiment, the hub ports 116a-f comprise a first port type, and the hub lead connector port 117 may comprise a second port type. The first port type and the second port type may be the same, or the first port type and the second port type may be different.

As shown in FIG. 3A, in one embodiment, the one or more transducer subarray 108 of the transducer array 100 comprises a support layer 156, an additional electrode (i.e., one or more electrode element 160), and a subarray port 164. The support layer 156 has a support layer peripheral edge 168, wherein the subarray port 164 extends from the support layer peripheral edge 168 and is supported by the support layer 156. In one embodiment, the electrode element 160 may be disposed within the support layer peripheral edge 168 and supported by the support layer 156.

In one embodiment, each of the one or more transducer subarray 108 has an area dependent on the support layer 156. For example, the support layer 156 of the first transducer subarray 108a has a first area, the support layer 156 of the second transducer subarray 108b has a second area, and the support layer 156 of the third transducer subarray 108c has a third area. While the first area, the second area, and the third area are shown as being different, in other embodiments, one or more of the first area, the second area, and the third area may be the same. For example, the first area of the support layer 156 of the first transducer subarray 108a is shown as being larger than third area of the support layer 156 of the third transducer subarray 108c, while, the first area of the support layer 156 of the first transducer subarray 108a is shown smaller than the second area of the support layer 156 of the third transducer subarray 108c.

In one embodiment, each of the one or more transducer subarray 108 has a shape dependent on the support layer 156. For example, the support layer 156 of the first transducer subarray 108a has a first shape, the support layer 156 of the second transducer subarray 108b has a second shape, and the support layer 156 of the third transducer subarray 108c has a third shape. While the first shape, the second shape, and the third shape are shown as being different, in other embodiments, one or more of the first shape, the second shape, and the third shape can be congruent, e.g., the same. In the example shown, the one or more transducer subarrays 108 are provided with a generally triangular shape, resembling a petal of a flower. However, it should be understood that the one or more transducer subarrays 108 can be provided with a shape having any suitable geometry, including, but not limited to: circular, oval, square, rectangular, polygonal, and fanciful shape, or truncated versions thereof, or a combination thereof.

In some embodiments, the support layer 156 of the one or more transducer subarray 108 may include a subarray neck 172 extending between the subarray port 164 and the electrode element 160 as shown by the subarray neck 172 of the second transducer subarray 108b. The subarray neck 172 may vary in length to allow the user to position the electrode element 160 of the one or more transducer subarray 108 closer to or further from the hub 104, as desired.

In one embodiment, the support layer 156 of the one or more transducer subarray 108 is constructed of a flexible material such that the one or more transducer subarray 108 is operable to contour to the patient's body. For example, when the transducer subarray 108 is placed on the patient's skin, the transducer subarray 108 is operable to contour (or conform) to the target area.

In one embodiment, the subarray port 164 may be constructed in accordance with and complementary to (e.g., is operable to be coupled to) the hub ports 116, as described above.

In one embodiment, the one or more transducer subarray 108 may be fixedly attached or selectively connected to the hub 104 by joining the subarray port 164 with a particular hub port 116 of the hub ports 116.

In one embodiment, the subarray port 164 may be electrically coupled to one of the hub ports 116 and configured to receive the TTSignal from the hub port 116. In one embodiment, the one or more transducer subarray 108 includes a flexible wire 176 extending through the subarray neck 172 electrically coupling the subarray port 164 to the electrode element 160, thereby providing the electrode element 160 with the TTSignal.

Referring now to FIG. 3B, shown therein is a cross-sectional diagram of an exemplary embodiment of the hub 104 constructed in accordance with the present disclosure. The first surface 120 may include a first portion 140 and a second portion 144, the second portion 144 being different from the first portion 140. As shown, the first portion 140 comprises a central surface area of the first surface 120 while the second portion 144 comprises an outer surface area of the first surface 120. In other embodiments, however, the first portion 140 may comprise a first half of the surface area of the first surface 120, while the second portion 144 may comprise a second half of the surface area of the first surface 120. In one embodiment, the second portion 144 is not contiguous. In other words, the first portion 140 may be disposed within the second portion 144 such that a first area of the second portion 144 is not contiguous with a second area of the second portion 144.

In some embodiments, the hub 104 may include an adhesive layer 148 on the first surface 120. The adhesive layer 148 may be disposed on the first portion 140 of the first surface 120, the second portion 144 of the first surface 120, or both. The adhesive layer 148 may be biocompatible with the patient to reduce interactions between the adhesive layer 148 and the patient's skin. In one embodiment, the adhesive layer 148 is an adhesive tape operable to hold the hub 104 in place on the patient as the patient goes about their daily activities.

In one embodiment, the hub 104 may further comprise an electrode element 160 (discussed in more detail below) disposed on or within the first portion 140 of the first surface 120 of the housing 112. With the electrode element 160 disposed on the first portion 140, the adhesive layer 148 would only be disposed on the second portion 144 of the first surface 120. (In FIG. 3B, for clarity, adhesive layer 148 is only shown on one area of the second portion 144, but in other embodiments the adhesive layer 148 could be in a continuous area around the electrode element 160—i.e., showing on both sides of the first portion 140 in FIG. 3B). The electrode element 160 is electrically coupled to a hub port (e.g., a hub port 116, or the hub lead connector port 117) and configured to receive the TTSignal(s).

Referring now to FIG. 4, shown therein is a cross-sectional view of an exemplary embodiment of the electrode element 160 of the first transducer subarray 108a along the line 4-4′ of FIG. 3A. The electrode element 160 generally comprises an electrode 180, electrically coupled to the subarray port 164 via the flexible wire 176 (shown in FIG. 3A), and a skin-interface layer 184. In one embodiment, the electrode element 160 further comprises a non-conducting layer 188 disposed such that the electrode 180 is interposed between the non-conducting layer 188 and the skin-interface layer 184. In some embodiments, the electrode element 160 further (optionally) comprises a dielectric layer 192 interposed between the electrode 180 and the skin-interface layer 184.

In one embodiment, the electrode 180 has a first electrode surface 196 on a skin-facing side 200 (e.g., as a first skin-facing surface), and a second electrode surface 204 on an opposing outwardly-facing side 208. The electrode 180 may be constructed of any conductive material having desirable properties such as, but not limited to, high conductivity, strong biocompatibility, and low reactivity with other layers or components of the electrode element 160. In one embodiment, the conductive material of the electrode 180 is selected from one or more of the following: silver, gold, tin, aluminum, titanium, platinum, stainless steel, carbon, copper, an alloy thereof, and/or some combination thereof.

The skin-interface layer 184 may have a first interface surface 212 disposed on the skin-facing side 200 of the electrode 180 and may be electrically connected to (i.e., in electrical communication with) the electrode 180. The skin-interface layer 184 may have a second interface surface 216 configured to be in contact with the patient's skin.

In one embodiment, the skin-interface layer 184 comprises one or more material configured to be one or more of electrically conductive, biocompatible when in contact with the patient's skin for an extended period of time, e.g., from 3 hours to a week at a time, flexible so as to not impede movement of the patient while the one or more transducer subarray 108 is in place, and resistant to movement on the patient's skin as the patient goes about their daily routine. In one embodiment, the skin-interface layer 184 is a gel, a hydrogel, a conductive gel or hydrogel, a polymerized gel or hydrogel, or a conductive polymerized gel or hydrogel, constructed in accordance with the gel/hydrogel layers described in U.S. Patent Publication No. 2021/0346693 A1, published Nov. 11, 2021 and entitled “CONDUCTIVE PAD GENERATING TUMOR TREATING FIELD AND METHODS OF PRODUCTION AND USE THEREOF” and U.S. Pat. No. 11,458,298, issued on Oct. 4, 2022, and entitled “ASSEMBLIES CONTAINING TWO CONDUCTIVE GEL COMPOSITIONS AND METHODS OF PRODUCTION AND USE THEREOF”, the entire contents of which are hereby incorporated herein in their entirety. In another embodiment, the skin-interface layer 184 is an adhesive or a conductive adhesive. In some embodiments, the skin-interface layer 184 extends (laterally) as far as the electrode 180 extends, whereas in other embodiments, the skin-interface layer 184 extends at least to the support layer peripheral edge 168 of the electrode element (FIG. 3A) or beyond.

The non-conducting layer 188 may have a first layer surface 209 disposed on the opposing outwardly-facing side 208 of the electrode 180. The non-conductive layer 188 may have a second layer surface 210 opposite of the first layer surface 209. The non-conducting layer 188 may be present as the support layer 156 (FIG. 3A) of the transducer subarray 108, and the electrode element 160 may be fixedly attached to the support layer 156. In one embodiment, the non-conducting layer 188 may be constructed of a durable, non-conductive material, such as a non-conductive fabric. In some embodiments, the non-conductive fabric may have a plurality of perforations that allow thermal dissipation from the electrode element 160.

In one embodiment, the non-conducting layer 188 may include an adhesive (constructed in accordance with the adhesive layer 148 discussed above) on the first layer surface 209. Further, the non-conducting layer 188 may be configured to cover and extend beyond the electrode element 160 such that the adhesive on the first layer surface 209 of the non-conducting layer 188 may contact the patient's skin to adhere the electrode element 160, and/or the transducer subarray 108, to the patient.

In one embodiment, the dielectric layer 192 may be constructed of a dielectric material so as to capacitively couple the electrode 180 with the skin of the patient. The dielectric material may take the form of a ceramic material or a high dielectric polymer. In one embodiment, the dielectric layer 192 may be present as the support layer 156. For example, when the dielectric layer 192 is not present, the first interface surface 212 of the skin-interface layer 184 may be in contact with the first electrode surface 196 on the skin-facing side 200 of the electrode 180.

In one embodiment, the electrode 180 and/or the dielectric layer 192 may (optionally) comprises a pathway 220 extending therethrough. The pathway 220 may be operable to allow moisture and/or air to move between the skin-interface layer 184 and an atmosphere on the second layer surface 210 of the non-conducting layer 188.

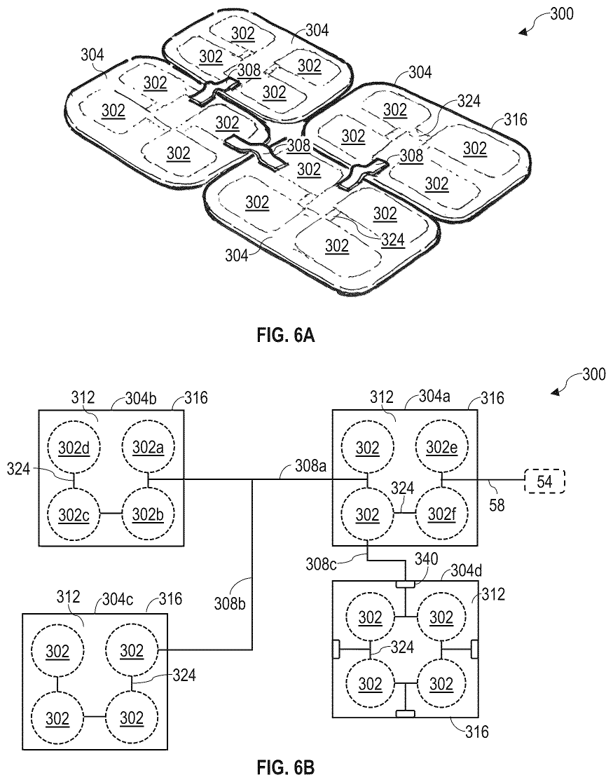

Referring now to FIG. 5, shown therein is a front view of an exemplary embodiment of the electronic apparatus 50 (FIG. 2) constructed and used in accordance with the present disclosure. As shown in FIG. 5, the first transducer array 70a and the second transducer array 70b (shown as constructed in accordance with the transducer array 100 (FIG. 3A), but may be constructed in accordance with the transducer arrays 300 (FIGS. 6A-6B), below) are attached to the chest of a patient 224 and are connected to the electric field generator 54 by the first conductive lead 58a and the second conductive lead 58b, respectively. In one embodiment, each component of the transducer array 70 (e.g., of the first transducer array 70a or of the second transducer array 70b) is configured to conform to a shape of the target area to provide the desired TTField.

For example, the transducer array 70 may be configured to prevent overlap between the transducer array 70 and one or more avoidance areas 232 (e.g., shown as a first avoidance area 232a and a second avoidance area 232b) on the body of the patient 224. For example, the first transducer array 70a is positioned on the right chest area and configured such that the first transducer array 70a does not include the transducer subarray 108 connected to the hub port 116d, so as to not interfere and/or overlap with the first avoidance area 232a, (i.e., a chemo port) located toward the top of the patient's right chest. Similarly, the second transducer array 70b is positioned on the patient's left chest area and configured such that the second transducer array 70b does not include the transducer subarray 108 connected to the hub port 116e to evade the second avoidance area 232b on the patient's body, e.g., a sensitive or highly innervated area of the patient's body.

As discussed above, the transducer array 70 selectively includes the one or more transducer subarrays 108 of varied area and shape to create a customized transducer array that generates the TTField(s) of a desired configuration, while bypassing the one or more avoidance areas 232 on the patient's body. In the example shown, the first transducer array 70a and the second transducer array 70b (constructed in accordance with the transducer array 100 described above) each include five of the one or more transducer subarrays 108, wherein two transducer subarrays 108 have a smaller area with the subarray neck 172 being shorter, one transducer subarray 108 has a larger area with the subarray neck 172 being shorter, and two transducer subarrays 108 have a larger area with the subarray neck 172 being longer. In one embodiment, the first transducer array 70a may include a transducer subarray 108 having a smaller size and smaller area, e.g., similar to the third transducer subarray 108c (FIG. 3A), coupled to the hub port 116d if the size and shape of the third transducer subarray 108c (FIG. 3A), when coupled to the first transducer array 70a, would not interfere with the first avoidance area 232a.

Turning now to FIGS. 6A-6B in combination, shown in FIG. 6A is a perspective view of an exemplary embodiment of a transducer array 300 and shown in FIG. 6B is a diagram of another embodiment of a transducer array 300 constructed in accordance with the present disclosure. The transducer array 300 may be a particular embodiment of the transducer array 70 (FIG. 2). The transducer array 300 comprises a plurality of electrode elements 302a-n and two or more transducer subarrays 304a-n, each comprising a subset of the plurality of electrode elements 302 and electrically coupled to at least one other of the n transducer subarrays by one or more flexible cable 308 (e.g., flexible cables 308a-c in FIG. 6B) and electrically coupled to the electric field generator 54 via the conductive lead 58.

Each transducer subarray 304 of the plurality transducer subarrays 304a-n comprises the subset of the plurality of electrode elements 302 and may include a support layer 312 that has a support layer peripheral edge 316 and that supports the subset of the plurality of electrode elements 302. Each electrode element 302 is constructed in accordance with the electrode element 160 described above and shown in FIG. 4. The subset of the plurality of electrode elements 302 within a particular transducer subarray 304 are interconnected via a flex wire 324. The flex wire 324 may be configured to distribute the TTSignal(s) to all of the electrode elements 302 of the subset of the particular transducer subarray 304 in a parallel circuit.

In one embodiment, the flex wire 324 may be configured to electrically couple each of the plurality of electrode elements 302 in a parallel circuit, a series circuit, or combination thereof. While the transducer array 300 is shown with each subset of the transducer subarray 304 having four electrode elements 302, it will be understood that each subset of transducer subarrays 304 may include as few as one electrode element 302 or as many electrode elements 302 as are required to provide the desired TTField or able to be supported by the support layer 312. In one embodiment, the flex wire 324 is configured to electrically couple at least one of the electrode elements 302 in parallel with at least one other electrode element 302. For example, a second electrode element 302b and a third electrode element 302c may be electrically coupled in series (as an electrode element pair) while a first electrode element 302a may be electrically coupled in parallel with the electrode element pair.

The one or more flexible cable 308 may be utilized to electrically couple multiples of the plurality of transducer subarrays 304 together. Exemplary embodiments of the flexible cable 308 may be constructed in accordance with any of the embodiments of the wires disclosed in U.S. application Ser. No. 17/490,120, as cited above. Each flexible cable 308 may be configured to be either fixedly or selectively attached to at least some portion of the flex wire 324, at least one of the plurality of electrode elements 302, another one of the one or more flexible cable 308, or a port 340. In one embodiment, the flexible cables 308 are constructed of a flexible and conductive material such that the transducer array 300 is operable to contour to the patient's body. For example, when the transducer array 300 is placed on the patient's skin, the flexible cables 308 may flex between each of the transducer subarrays 304 thereby further enabling the transducer array 300 to contour (or conform) to the target area.

For example, in one embodiment, a first flexible cable 308a may be attached to the flex wire 324 of the first transducer subarray 304a and the flex wire 324 of the second transducer subarray 304b. A second flexible cable 308b may be attached to the first flexible cable 308a (or the flex wire 324 of the first transducer subarray 304a) and a particular electrode element 302 of the third transducer subarray 304c. In this configuration, the second transducer subarray 304b is electrically coupled in parallel with the third transducer subarray 304c. Further, in one embodiment, at least one electrode element 302 (e.g., the first electrode element 302a) is electrically coupled in parallel with at least one other electrode element 302 (e.g., the electrode elements 302b-d). In one embodiment, because the electrode elements 302 are not electrically coupled in series with each of the other electrode elements 302, the flex wire 324 and the flexible cable 308 do not need to be constructed to carry a current or a voltage for all electrode elements 302, thereby increasing flexibility and decreasing weight of the transducer array 300 thus improving comfortability for the patient.

The support layer 312 may be constructed and function in accordance with the support layer 156, as described above. In one embodiment, an area and a shape of each of the transducer subarray 304 of the plurality of transducer subarrays 304a-n will be determined by a number and arrangement of the subset of the plurality of electrode elements 302 contained within each transducer subarray 304.

In the example shown in FIG. 6B, the transducer array 300 includes four transducer subarrays 304a-d, each transducer subarray 304 having a subset of the plurality of electrode elements 302, the subset having four electrode elements 302. Within each of the plurality of transducer subarrays 304, the electrode elements 302 are interconnected via flex wires 324. The TTSignal is supplied to at least one of the transducer subarrays 304 via the conductive lead 58, which is connected to the electric field generator 54. For example, the conductive lead 58 is attached to the flex wires 324 that connects the two electrode elements 302 of the first transducer subarray 304a (a fifth electrode element 302e and a sixth electrode element 302f).

While the example shown in FIG. 6B depicts each of the transducer subarrays 304 as having a square shape with four electrode elements 302, it will be understood that the transducer subarray 304 may comprise other shapes and number of electrode elements 302. The shape and number of electrode elements 302 may vary between each transducer subarray 304 of the transducer array 300. The specific shape, size, and positioning of each transducer subarray 304 will be selected so as to generate the TTField of a desired configuration, direction, and intensity at the treatment area and only at that treatment area so as to focus the treatment. In one embodiment, the specific shape and size of each transducer subarray 304 may be selected such that a size of the transducer array 300 is about 200 cm².

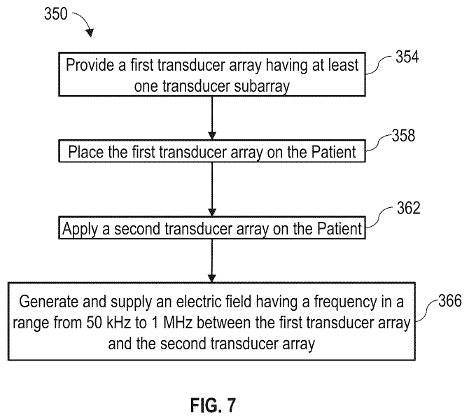

Referring now to FIG. 7, shown therein is a process flow diagram of an exemplary embodiment of a process 350 of using the electronic apparatus 50 (FIG. 2) and the transducer array(s) 70 (e.g., the transducer array 100 and/or the transducer array 300) in accordance with the present disclosure. The process 350 generally comprises: providing a first transducer array 70a having at least one transducer subarray 108 or 304 (step 354); placing the first transducer array 70a on the patient at a designated location (step 358); applying a second transducer array 70b on the patient (step 362); and generating and supplying an electric field having a frequency in a range from 50 kHz to 1 MHz between the first transducer array 70a and the second transducer array 70b (step 366).

In one embodiment, providing the first transducer array 70a having at least one transducer subarray 108 or 304 (step 354) may include providing one of the transducer array 100 or the transducer array 300 as the first transducer array 70a having the at least one transducer subarray 108 or 304. In one embodiment, providing the first transducer array 70a having the at least one transducer subarray 108 or 304 includes providing a skin-interface layer 184 either on the patient or on the first electrode surface 196 on a skin-facing side 200 of the electrode 180. In another embodiment, the skin-interface layer 184 may be provided either on the patient or on the skin-facing surface of the dielectric layer 192, forming a first interface surface 212 between the dielectric layer 192 and the skin-interface layer 184.

In one embodiment, placing the first transducer array 70a on the patient at a designated location (step 358) includes placing the first transducer array 70a having one or more transducer subarray 108 or 304 on the patient at the designated location on the patient associated with providing a therapeutic benefit to treat the patient at the target region.

In one embodiment, applying the second transducer array 70b on the patient (step 362) includes placing the second transducer array 70b having one or more transducer subarray 108 or 304 on the patient, as described above for the first transducer subarray 70a. In some embodiments, the second designated location may be a second location on the patient associated with providing a therapeutic benefit to treat the patient at the target region.

In one embodiment, generating and supplying the electric field having the frequency in the range from 50 kHz to 1 MHz between the first transducer array 70a and the second transducer array 70b (a first pair of transducer arrays; step 366) includes supplying a first alternating current waveform to the first transducer array 70a for a first period of time and supplying a second alternating current waveform to the second transducer array 70b for a second period of time. The first transducer array 70a and the second transducer array 70b, being supplied with the electric signals, causes an electrical current to flow between the first transducer array 70a and the second transducer array 70b. The electrical current generates an electric field (i.e., TTField), having a frequency and an amplitude, to be generated between the first transducer array 70a and the second transducer array 70b (the first pair of transducer arrays). In an embodiment, a second pair of transducer arrays can be positioned to similarly induce an electric field through the same target area, but in a different direction (e.g., a direction perpendicular to the first electric field between the first pair of transducer arrays). In one embodiment, generating and supplying TTFields having the frequency in the range from 50 kHz to 1 MHz to a target area includes generating an electric field through the target area between the first pair of transducer arrays 70 for a first period of time and generating an electric field through the target area between the second pair of transducer arrays 70 for a second period of time, and then repeating these two steps in a cycle.

In one embodiment, the first period of time may be of the same or a similar duration to the second period of time whereas in other embodiments, the first period of time may be of a different duration to the second period of time. Additionally, the first period of time may or may not overlap with the second period of time.

Certain non-limiting embodiments of the present disclosure are related to kits that include any of the components of the electronic apparatus 50 as described above, such as, but not limited to, one or more transducer arrays 70, 100, 300 in combination with one or more component, device, and/or element utilized in accordance with this disclosure or otherwise contemplated herein. The kits may optionally further include one or more of any of the optional components disclosed or otherwise contemplated herein. The kits may optionally further include one or more devices (or one or more components of devices) utilized in one or more additional therapy steps.

In one embodiment, the kit may further include instructions for performing any of the processes or methods disclosed or otherwise contemplated herein. For example (but not by way of limitation), the kit may include instructions for applying one or more components of the electronic apparatus 50 to the skin of the patient, instructions for applying the alternating electric field to the patient, and/or instructions for when to activate and turn off the alternating electric field in relation to the administration of the TTFields.

In addition to the components described in detail herein above, the kits may further contain other component(s)/reagent(s) for performing any of the particular methods described or otherwise contemplated herein. For example (but not by way of limitation), the kits may additionally include: (i) components for preparing the skin prior to application of the skin-interface layer 184 and/or transducer arrays 70, 100, 300 thereon (i.e., a razor, a cleansing composition or wipe/towel, etc.); (ii) components for removal of the skin-interface layer/transducer array(s); (iii) components for cleansing of the skin after removal of the skin-interface layer/transducer array(s); (iv) components for isolation of the cancer cells/portion of tumor; (v) components for irradiation of the isolated, alternating electric field-treated cancer cells/portion of tumor; and/or (vi) components for isolation of the irradiated, alternating electric field-treated cancer cells. The nature of these additional component(s)/reagent(s) will depend upon the particular treatment format, and identification thereof is well within the skill of one of ordinary skill in the art; therefore, no further description thereof is deemed necessary. Also, the components/reagents present in the kits may each be in separate containers/compartments, or various components/reagents can be combined in one or more containers/compartments, depending on the sterility, cross-reactivity, and stability of the components/reagents.

The kit may be disposed in any packaging that allows the components present therein to function in accordance with the present disclosure. In certain non-limiting embodiments, the kit further comprises a sealed packaging in which the components are disposed. In certain particular (but non-limiting) embodiments, the sealed packaging is substantially impermeable to air and/or substantially impermeable to light.

In addition, the kit can further include a set of written instructions explaining how to use one or more components of the kit. A kit of this nature can be used in any of the methods described or otherwise contemplated herein.

In certain non-limiting embodiments, the kit has a shelf life of at least about six months, and between six months and 24 months.

### Illustrative Embodiments

The following is a non-limiting list of illustrative embodiments of the inventive concepts disclosed herein:

Illustrative Embodiment 1. A transducer array, comprising: a hub comprising a housing having a housing peripheral edge having a boundary, and a hub port supported by the housing, the hub port positioned within the boundary of the housing peripheral edge; and a transducer subarray comprising an electrode, a subarray port, and a support layer having a support layer peripheral edge and supporting the electrode and the subarray port, the subarray port coupling the hub port and the electrode, the electrode receiving an alternating current waveform having a frequency in a range between 50 kHz-1 MHz from the hub via the subarray port.

Illustrative Embodiment 2. The transducer array of Illustrative Embodiment 1, wherein the hub further comprises a hub lead connector port supported by the housing, and the transducer array further comprising: a conductive lead electrically coupled to the hub lead connector port.

Illustrative Embodiment 3. The transducer array of Illustrative Embodiment 1, wherein the subarray port of the transducer subarray is disposed at least partially within the support layer peripheral edge.

Illustrative Embodiment 4. The transducer array of Illustrative Embodiment 1, wherein the electrode is a first electrode, the hub port is a first hub port, and wherein the hub further comprises a second hub port supported by the housing, the second hub port positioned within the boundary of the housing peripheral edge, wherein the transducer subarray is a first transducer subarray, the subarray port is a first subarray port, the support layer is a first support layer, and the support layer peripheral edge is a first support layer peripheral edge, and further comprising a second transducer subarray, the second transducer subarray comprising a second electrode, a second subarray port, a second support layer having a second support layer peripheral edge and supporting the second electrode and the second subarray port, the second subarray port coupling the second hub port and the second electrode, the second electrode receiving the alternating current waveform from the hub via the second subarray port.

Illustrative Embodiment 5. The transducer array of Illustrative Embodiment 4, wherein the first support layer peripheral edge of the first transducer subarray has a first shape and the second support layer peripheral edge of the second transducer subarray has a second shape different from the first shape.

Illustrative Embodiment 6. The transducer array of Illustrative Embodiment 4, wherein the first support layer peripheral edge of the first transducer subarray has a first shape and the second support layer peripheral edge of the second transducer subarray has a second shape congruent with the first shape.

Illustrative Embodiment 7. The transducer array of Illustrative Embodiment 4, wherein the first support layer of the first transducer subarray has a first area and the second support layer of the second transducer subarray has a second area different from the first area.

Illustrative Embodiment 8. The transducer array of Illustrative Embodiment 4, wherein the first support layer of the first transducer subarray has a first area and the second support layer of the second transducer subarray has a second area congruent with the first area.

Illustrative Embodiment 9. The transducer array of Illustrative Embodiment 2, wherein the conductive lead comprises a first end and a second end, the second end of the conductive lead being operable to be selectively coupled to the hub lead connector port.

Illustrative Embodiment 10. The transducer array of Illustrative Embodiment 1, wherein the transducer subarray comprises more than one electrode, each electrode being configured to receive the alternating current waveform.

Illustrative Embodiment 11. The transducer array of Illustrative Embodiment 1, wherein the housing of the hub further comprises a first surface and a second surface, the hub further comprising an additional electrode disposed on at least a first portion of the first surface of the housing and being configured to receive the alternating current waveform.

Illustrative Embodiment 12. The transducer array of Illustrative Embodiment 11, wherein the hub further comprises an adhesive layer disposed on a second portion of the first surface of the housing, the second portion being different from the first portion.

Illustrative Embodiment 13. The transducer array of Illustrative Embodiment 1, wherein the housing of the hub further comprises a first surface and a second surface, the hub further comprising an adhesive layer disposed on the first surface of the housing.

Illustrative Embodiment 14. The transducer array of Illustrative Embodiment 1, wherein the electrode further comprises a first skin-facing surface and a second surface opposite the first skin-facing surface, and further comprising a skin-interface layer, the skin-interface layer being disposed on the first skin-facing surface of the electrode.

Illustrative Embodiment 15. The transducer array of Illustrative Embodiment 14, further comprising a dielectric layer disposed between the skin-interface layer and the electrode.

Illustrative Embodiment 16. The transducer array of Illustrative Embodiment 14, further comprising a layer of anisotropic material disposed between the skin-interface layer and the electrode.

Illustrative Embodiment 17. The transducer array of Illustrative Embodiment 15, further comprising a layer of anisotropic material disposed between the skin-interface layer and the dielectric layer.

Illustrative Embodiment 18. The transducer array of Illustrative Embodiment 1, wherein a number of hub ports, n, are distributed around the housing peripheral edge equidistant or approximately equidistant from each other, and n transducer subarrays are each coupled to a respective one of the n hub ports via a respective subarray port.

Illustrative Embodiment 19. The transducer array of Illustrative Embodiment 1, wherein a number of hub ports, n, are distributed around the housing peripheral edge equidistant or approximately equidistant from each other, and n-1 or n-2 transducer subarrays are each coupled to a respective one of the n hub ports via a respective subarray port.

Illustrative Embodiment 20. A transducer array, comprising:
a plurality of electrode elements, each electrode element comprising an electrode configured for placement on a body of a patient and to receive an alternating current waveform;
a first transducer subarray comprising a first support layer having a first support layer peripheral edge and supporting a first subset of the plurality of electrode elements operable to receive the alternating current waveform;
a second transducer subarray comprising a second support layer having a second support layer peripheral edge and supporting a second subset of the plurality of electrode elements operable to receive the alternating current waveform, the second support layer peripheral edge being disposed apart from the first support layer peripheral edge; and
a flexible cable electrically coupled to the first subset of the plurality of electrode elements and operable to transmit the alternating current waveform to the second subset of the plurality of electrode elements.

Illustrative Embodiment 21. The transducer array of Illustrative Embodiment 20, wherein the alternating current waveform has a frequency in a range between 50 kHz-1 MHz.

Illustrative Embodiment 22. The transducer array of Illustrative Embodiment 20, wherein each electrode is configured for placement on a body of a patient and to receive the alternating current waveform having a frequency in a range between 50 kHz-1 MHz.

Illustrative Embodiment 23. The transducer array of Illustrative Embodiment 20, further comprising a conductive lead configured to receive the alternating current waveform having a frequency in a range between 50 kHz-1 MHz and electrically coupled to the flexible cable.

Illustrative Embodiment 24. The transducer array of Illustrative Embodiment 23, wherein the flexible cable is a first flexible cable, and further comprising:
a third transducer subarray comprising a third support layer having a third support layer peripheral edge and supporting a third subset of the plurality of electrode elements; and
a second flexible cable operable to electrically couple the first subset of the plurality of electrode elements and the third subset of the plurality of electrode elements.

Illustrative Embodiment 25. The transducer array of Illustrative Embodiment 24, wherein the conductive lead is electrically coupled to the second flexible cable or to the first transducer subarray.

Illustrative Embodiment 26. The transducer array of Illustrative Embodiment 24, wherein the conductive lead is electrically coupled to the first flexible cable.

Illustrative Embodiment 27. The transducer array of Illustrative Embodiment 20, wherein the first subset of the plurality of electrode elements includes at least two electrode elements.

Illustrative Embodiment 28. The transducer array of Illustrative Embodiment 27, wherein the at least two electrode elements are electrically coupled to the flexible cable and are electrically disposed in parallel with each other.

Illustrative Embodiment 29. The transducer array of Illustrative Embodiment 24, comprising four or more subarrays, each in electrical communication with the conductive lead and each electrically coupled to at least one other subarray via one or more flexible cable.

Illustrative Embodiment 30. A transducer array system, comprising:
an electric field generator operable to generate an electrical signal having an alternating current waveform at a frequency in a range from 50 kHz to 1 MHZ;
a plurality of electrode elements, each electrode element comprising an electrode configured for placement on a body of a patient and to receive an alternating current waveform having a frequency in a range between 50 kHz-1 MHZ;
a first transducer subarray comprising a first support layer having a first support layer peripheral edge and supporting a first subset of the plurality of electrode elements operable to receive the alternating current waveform; and
a second transducer subarray comprising a second support layer having a second support layer peripheral edge and supporting a second subset of the plurality of electrode elements operable to receive the alternating current waveform;
wherein the first support layer is different from the second support layer and the first transducer subarray is electrically disposed in parallel with the second transducer subarray.

Illustrative Embodiment 31. The transducer array system of Illustrative Embodiment 30, further comprising: a hub comprising a housing having a housing peripheral edge having a boundary, and a first port and a second port supported by the housing, the first port and the second port positioned within the boundary of the housing peripheral edge; wherein the first transducer subarray is coupled to the first port and the second transducer subarray is coupled to the second port.

Illustrative Embodiment 32. The transducer array system of Illustrative Embodiment 31, further comprising one or more additional transducer subarrays, each comprising a respective support layer having a respective support layer peripheral edge and supporting an additional subset of the plurality of electrode elements operable to receive the alternating current waveform; and wherein each additional transducer subarray is coupled to a respective port supported by the housing of the hub.

Illustrative Embodiment 33. A transducer array system, comprising:
a conductive lead;
a plurality of transducer subarrays, each of the plurality of transducer subarrays comprising an electrode, one or more ports, and a support layer having a support layer peripheral edge and supporting the electrode and the one or more ports, the electrode being configured to receive an alternating current waveform having a frequency in a range between 50 kHz-1 MHz for a period of time, the one or more ports disposed at least partially within the support layer peripheral edge; and
a plurality of flexible cables electrically coupled to the conductive lead and each flexible cable operable to connect to at least one of the one or more ports and electrically couple any two transducer subarrays of the plurality of transducer subarrays; and
wherein a first transducer subarray of the plurality of transducer subarrays is electrically coupled to the conductive lead and electrically disposed between at least two other transducer subarrays of the plurality of transducer subarrays.

Illustrative Embodiment 34. The transducer array system of Illustrative Embodiment 33, wherein at least one of the one or more ports are configured to be selectively coupled to the conductive lead.

Illustrative Embodiment 35. The transducer array system of Illustrative Embodiment 33, wherein the transducer array system further comprises a hub and the conductive lead is configured to be selectively coupled to the hub.

Illustrative Embodiment 36. A kit, comprising: a hub comprising a housing having a housing peripheral edge having a boundary, and a first port supported by the housing, the first port positioned within the boundary of the housing peripheral edge of the housing; and a transducer subarray comprising an electrode, a second port, and a support layer having a support layer peripheral edge and supporting the electrode and second port, the second port operable to be coupled to the first port, the electrode receiving an alternating current waveform having a frequency in a range between 50 kHz-1 MHz from the second port.

Illustrative Embodiment 36. The kit of Illustrative Embodiment 36, further comprising a skin-interface layer operable to be disposed between the transducer subarray and a patient's skin.

Illustrative Embodiment 38. The kit of Illustrative Embodiment 36, further comprising written instructions explaining the process 350.

Illustrative Embodiment 39. The kit of Illustrative Embodiment 36, further comprising components for preparing a patient's skin.

Even though particular combinations of features and steps are recited in the claims, Illustrative embodiments, and/or disclosed in the specification, these combinations are not intended to limit the disclosure. In fact, many of these features and steps may be combined in ways not specifically recited in the claims, Illustrative embodiments, and/or disclosed in the specification. Although each dependent claim listed below may directly depend on only one other claim, the disclosure includes each dependent claim in combination with every other claim in the claim set.

Similarly, although each illustrative embodiment listed above may directly depend on only one other illustrative embodiment, the disclosure includes each illustrative embodiment in combination with every other illustrative embodiment in the set of illustrative embodiments for each mode of the inventive concepts disclosed herein.

What is claimed is:

1. A transducer array, comprising:
a hub comprising a housing having a housing peripheral edge having a boundary, and a hub port supported by the housing, the hub port positioned within the boundary of the housing peripheral edge; and
a transducer subarray comprising an electrode, a subarray port, and a support layer having a support layer peripheral edge and supporting the electrode and the subarray port, the subarray port coupling the hub port and the electrode, the electrode receiving an alternating current waveform having a frequency in a range between 50 kHz-1 MHz from the hub via the subarray port.

2. The transducer array of claim 1, wherein the hub further comprises a hub lead connector port supported by the housing, and the transducer array further comprising: a conductive lead electrically coupled to the hub lead connector port.

3. The transducer array of claim 1, wherein the subarray port of the transducer subarray is disposed at least partially within the support layer peripheral edge.

4. The transducer array of claim 1, wherein the electrode is a first electrode, the hub port is a first hub port, and wherein the hub further comprises a second hub port supported by the housing, the second hub port positioned within the boundary of the housing peripheral edge, wherein the transducer subarray is a first transducer subarray, the subarray port is a first subarray port, the support layer is a first support layer, and the support layer peripheral edge is a first support layer peripheral edge, and further comprising a second transducer subarray, the second transducer subarray comprising a second electrode, a second subarray port, a second support layer having a second support layer peripheral edge and supporting the second electrode and the second subarray port, the second subarray port coupling the second hub port and the second electrode, the second electrode receiving the alternating current waveform from the hub via the second subarray port.

5. The transducer array of claim 4, wherein the first support layer peripheral edge of the first transducer subarray has a first shape and the second support layer peripheral edge of the second transducer subarray has a second shape different from the first shape.

6. The transducer array of claim 4, wherein the first support layer peripheral edge of the first transducer subarray has a first shape and the second support layer peripheral edge of the second transducer subarray has a second shape congruent with the first shape.

7. The transducer array of claim 4, wherein the first support layer of the first transducer subarray has a first area and the second support layer of the second transducer subarray has a second area different from the first area.

8. The transducer array of claim 4, wherein the first support layer of the first transducer subarray has a first area and the second support layer of the second transducer subarray has a second area congruent with the first area.

9. The transducer array of claim 2, wherein the conductive lead comprises a first end and a second end, the second end of the conductive lead being operable to be selectively coupled to the hub lead connector port.

10. The transducer array of claim 1, wherein the transducer subarray comprises more than one electrode, each electrode being configured to receive the alternating current waveform.

11. The transducer array of claim 1, wherein the housing of the hub further comprises a first surface and a second surface, the hub further comprising an additional electrode disposed on at least a first portion of the first surface of the housing and being configured to receive the alternating current waveform.

12. The transducer array of claim 1, wherein a number of hub ports, n, are distributed around the housing peripheral edge equidistant or approximately equidistant from each other, and n transducer subarrays are each coupled to a respective one of the n hub ports via a respective subarray port.

13. The transducer array of claim 1, wherein a number of hub ports, n, are distributed around the housing peripheral edge equidistant or approximately equidistant from each other, and n-1 or n-2 transducer subarrays are each coupled to a respective one of the n hub ports via a respective subarray port.

14. A transducer array, comprising:
a plurality of electrode elements, each electrode element comprising an electrode configured for placement on a body of a patient and to receive an alternating current waveform having a frequency in a range between 50 kHz-1 MHZ;
a first transducer subarray comprising a first support layer having a first support layer peripheral edge and supporting a first subset of the plurality of electrode elements operable to receive the alternating current waveform;
a second transducer subarray comprising a second support layer having a second support layer peripheral edge and supporting a second subset of the plurality of electrode elements operable to receive the alternating current waveform, the second support layer peripheral edge being disposed apart from the first support layer peripheral edge; and
a flexible cable electrically coupled to the first subset of the plurality of electrode elements and operable to transmit the alternating current waveform to the second subset of the plurality of electrode elements.

15. The transducer array of claim 14, wherein the flexible cable is a first flexible cable, and further comprising:
a third transducer subarray comprising a third support layer having a third support layer peripheral edge and supporting a third subset of the plurality of electrode elements; and
a second flexible cable operable to electrically couple the first subset of the plurality of electrode elements and the third subset of the plurality of electrode elements.

16. The transducer array of claim 15, wherein the conductive lead is electrically coupled to the second flexible cable or to the first transducer subarray.

17. The transducer array of claim 14, wherein the first subset of the plurality of electrode elements includes at least two electrode elements electrically disposed in parallel with each other.

18. A transducer array system, comprising:
an electric field generator operable to generate an electrical signal having an alternating current waveform at a frequency in a range from 50 kHz to 1 MHZ;
a plurality of electrode elements, each electrode element comprising an electrode configured for placement on a body of a patient and to receive an alternating current waveform having a frequency in a range between 50 kHz-1 MHZ;
a first transducer subarray comprising a first support layer having a first support layer peripheral edge and supporting a first subset of the plurality of electrode elements operable to receive the alternating current waveform;
a second transducer subarray comprising a second support layer having a second support layer peripheral edge and supporting a second subset of the plurality of electrode elements operable to receive the alternating current waveform; and
wherein the first support layer is different from the second support layer and the first transducer subarray is electrically disposed in parallel with the second transducer subarray.

19. The transducer array system of claim 18, further comprising:
a hub comprising a housing having a housing peripheral edge having a boundary, and a first port and a second port supported by the housing, the first port and the second port positioned within the boundary of the housing peripheral edge; and
wherein the first transducer subarray is coupled to the first port and the second transducer subarray is coupled to the second port.

20. The transducer array system of claim 19, further comprising one or more additional transducer subarrays, each comprising a respective support layer having a respective support layer peripheral edge and supporting an additional subset of the plurality of electrode elements operable to receive the alternating current waveform; and wherein each additional transducer subarray is coupled to a respective port supported by the housing of the hub.
